(19) United States
(12) Patent Application Publication
(10) Pub. No.: US 2024/0180619 A1
(43) Pub. Date: Jun. 6, 2024

(54) LASER TREATMENT DEVICE

(71) Applicant: Tag Dream Medical Ltd., Yokne'am lllit (IL)

(72) Inventors: Yuval PERETS, Moshav Beit Shearim (IL); Yaron Tal, Netanya (IL); Dan Nabel, Kiryat Tivon (IL); Dan Michael, Moshav Beit Shearim (IL)

(73) Assignee: Tag Dream Medical Ltd., Yokne'am lllit (IL)

(21) Appl. No.: 18/444,023

(22) Filed: Feb. 16, 2024

Related U.S. Application Data
(63) Continuation of application No. PCT/IL2022/050211, filed on Feb. 23, 2022.
Application No. PCT/IL2022/050211 is a continuation-in-part of application No. 17/405,617, filed on Aug. 18, 2021, now Pat. No. 11,369,398.
(63) Continuation-in-part of application No. 18/041,881, filed on Feb. 16, 2023.
(60) Provisional application No. 63/252,276, filed on Oct. 5, 2021.
(60) Provisional application No. 63/067,367, filed on Aug. 19, 2020.

(51) Int. Cl. A61B 18/28; A61B 18/00; A61B 18/22

(52) U.S. Cl. CPC A61B 18/28; A61B 2018/00095; A61B 2018/00101; A61B 2018/00702; A61B 2018/2272

(57) ABSTRACT

Apparatus is described including a tool, the tool including a shaft having a distal portion that is sized and shaped to be inserted into a subject during a surgical procedure. The tool includes a tissue-treatment tip, at the distal portion, that defines a beam deflector. An optical fiber is positioned to emit laser energy into the beam deflector. The beam deflector has a deflector surface, a first portion of which is shaped and positioned to reflect the laser energy toward a tissue-treatment tip surface, and a second portion of which is configured to absorb the laser energy and thermally conduct the absorbed energy to the tissue-treatment tip surface. The tissue-treatment tip surface is configured to thermally conduct the absorbed energy to the tissue by contacting the tissue. Other embodiments are also described.

### CROSS-REFERENCE TO RELATED APPLICATIONS

The present application is a continuation of International Application PCT/IL2022/050211, filed Feb. 23, 2022, which published as PCT Publication WO 2023/021497 to Perets et al., and which:
is a continuation-in-part of U.S. application Ser. No. 17/405,617, filed Aug. 18, 2021, entitled, “HYBRID LASER CUTTER,” now U.S. Pat. No. 11,369,398, and
claims the benefit of U.S. Provisional Application 63/252,276, filed Oct. 5, 2021, entitled, “HYBRID SURGICAL DEVICE.”

The present application is also a continuation-in-part of U.S. application Ser. No. 18/041,881, filed Feb. 16, 2023, which is the US national stage of International Application PCT/IL2021/051004, filed Aug. 18, 2021, which published as PCT Publication WO 2022/038604 to Perets et al., which claims the benefit of U.S. Provisional Application 63/067,368, filed Aug. 19, 2020, entitled, “HYBRID LASER CUTTER.”

Each of the above-mentioned applications is assigned to the assignee of the present application and is incorporated herein by reference.

### FIELD OF THE INVENTION

The present invention relates generally to surgical tools, and more particularly to minimally invasive surgical tools.

### BACKGROUND

U.S. Pat. No. 5,342,358 to Daikuzono describes apparatus that performs operations such as incision, coagulation and evaporation of the tissue of a living body such as human body with laser lights. An apparatus for performing a surgical operation of the tissue of a living body with laser lights while contacting a blade with the tissue of the living body has a holding portion which is held by an operator, a blade which is integral with the holding portion and is made of a material which generates heat on exposure to laser lights and cannot transmit the laser lights therethrough, and an optical fiber which receives laser lights for emitting the laser lights from the front end thereof, the blade being positioned in such a manner that a part of the blade is located in the irradiation area of the laser lights from the optical fiber and the optical fiber is movable toward and away from the blade while the optical fiber is held by the holding portion.

### SUMMARY OF THE INVENTION

In accordance with some applications of the present invention, a minimally invasive surgical tool is provided that comprises a tissue-treatment tip that absorbs laser energy (e.g., from an optical fiber), photothermally converts the laser energy into absorbed thermal energy, and uses the absorbed energy to treat tissue of a subject by conducting the absorbed energy from a tissue-treatment tip surface to the tissue, while the tissue-treatment tip surface is at an elevated temperature due to the absorbed energy.

The following is a non-limiting list of examples of target tissues of the subject and different types of surgeries in which the surgical tool may be used:
a meniscus of a knee of the subject, e.g., in a meniscectomy or partial meniscectomy,
cartilage debridement procedures,
microfracture procedures,
bone drilling,
tissue of the hip, e.g., in a hamstring repair, or gluteus medius repair,
spine decompression,
disc replacement,
tissue of the shoulder, e.g., in a shoulder synovectomy, frozen shoulder surgery,
arthroscopic capsular release, rotator cuff repair,
a tendon or ligament, e.g., for use in arthrolysis,
tissue of the bicep, e.g., in a biceps tenotomy,
hemorrhoid removal,
tissue of the hand, e.g., in carpel tunnel surgery,
removal of fecal impaction,
adenoidectomy,
hysteroscopic surgery, e.g., removal of pedunculated submucosal fibroids,
laparoscopic surgery, e.g., removal of subserosal fibroids,
abdominal surgery, e.g., performed laparoscopically,
cyst cutting, and
endoscopic stomach polyp removal.

For some applications, the tissue-treatment tip comprises a beam deflector into which an optical fiber emits the laser energy. The beam deflector has a deflector surface that absorbs the laser energy and thermally conducts the absorbed energy to the tissue-treatment tip surface. The tissue-treatment tip surface thermally conducts the absorbed energy to the tissue by contacting the tissue.

For some applications, the tissue-treatment tip uses the absorbed laser energy and a mechanical force to treat the tissue. The tissue-treatment tip treats the tissue by thermally conducting the absorbed energy to the tissue while a motor induces motion of the tissue-treatment tip.

For some such applications, the absorbed laser energy elevates the temperature of the tissue-treatment tip, reducing an amount of mechanical force that is required to treat the tissue.

For some such applications, the tissue-treatment tip defines a cavity, and a portion of the cavity absorbs the laser energy and thermally conducts the absorbed energy to a surface of the tissue-treatment tip while the motor induces motion of the tissue-treatment tip.

For some such applications, an optical fiber emits the laser energy into a beam deflector, and the tissue-treatment tip comprises a blade that absorbs reflected laser energy from the beam deflector, while the motor induces motion of the blade.

For some such applications, the tissue-treatment tip defines a blade and a thermal-treatment tip into which the laser energy is emitted. The tissue-treatment tip treats the tissue by: (i) a motor inducing motion of the blade, and (ii) thermally conducting the absorbed energy from the thermal-treatment tip to the tissue.

In accordance with some applications of the present invention, a method is provided for coupling an optical fiber to a surface of a metal component. While a distal end of the optical fiber is positioned facing the surface, laser energy is delivered through the optical fiber and is emitted to the surface. The laser energy elevates the temperature of fused silica of the optical fiber, melting the fused silica onto the surface and thereby coupling the distal end of the optical fiber to the surface.

For some applications, the melting point of the metal component is higher than the melting point of the fused silica, such that the fused silica melts, yet the surface does not melt.

For some applications, the fused silica is melted onto the surface so as to create a seal of fused silica that is impermeable to liquids involved in a surgical procedure.

For some applications, the coupled optical fiber and metal component are used to assemble a surgical device.

There is therefore provided, in accordance with an application of the present invention, apparatus for use in a surgical procedure, the apparatus including:
a tool including:
   a handle at a proximal region of the tool;
   an elongate shaft extending in a distal direction from the handle, the elongate shaft having proximal and distal portions, the distal portion of the shaft being sized and shaped to be inserted into a subject during a surgical procedure;
   a tissue-treatment tip disposed at the distal portion of the shaft, the tissue-treatment tip configured to contact tissue of the subject:
      (a) defining a tissue-treatment tip surface that is configured to face the tissue, and
      (b) being shaped to define a beam deflector having a deflector surface; and
   an optical fiber positioned to emit laser energy into the beam deflector, and:
a first portion of the deflector surface is shaped and positioned so as to reflect the laser energy toward the tissue-treatment tip surface,
a second portion of the deflector surface is configured to absorb the laser energy and thermally conduct the absorbed energy to the tissue-treatment tip surface, and
the tissue-treatment tip surface is configured to thermally conduct the absorbed energy to the tissue by contacting the tissue.

In an application:
the beam deflector is shaped to define a cavity,
the first portion of the deflector surface is a first portion of an internal surface of the cavity, and
the second portion of the deflector surface is a second portion of the internal surface of the cavity.

In an application, the first portion of the deflector surface is a reflective coating.

In an application, the second portion of the deflector surface includes tungsten.

In an application, the second portion of the deflector surface includes molybdenum.

In an application, the second portion of the deflector surface includes stainless steel.

In an application, the second portion of the deflector surface includes a cobalt-chrome alloy.

In an application, an angular orientation of the tissue-treatment tip is fixed with respect to an angular orientation of the handle.

In an application, a distance of the tissue-treatment tip from the handle is fixed.

In an application, the beam deflector includes an optical light guide.

In an application, the optical light guide includes sapphire or diamond.

In an application, the optical light guide includes a material having a melting point that is higher than 1700 degrees Celsius.

In an application, the optical fiber is a first optical fiber, and the tool includes a second optical fiber, each optical fiber being positioned to emit laser energy into a respective region of the beam deflector.

In an application, the tool is configured such that emitting laser energy into a respective part of the beam deflector causes:
a respective region of the first portion of the deflector surface to reflect the laser energy toward the tissue-treatment tip surface, and
a respective region of the second portion of the deflector surface to absorb the laser energy and thermally conduct the absorbed energy to a respective portion of the tissue-treatment tip surface.

In an application, the tool includes a controller, the controller being configured to separately control:
emission of laser energy from the first optical fiber, and
emission of laser energy from the second optical fiber.

In an application, the tissue-treatment tip surface includes a plurality of discrete tissue-contact elements, each of the tissue-contact elements defining a respective tissue-treatment tip surface.

In an application, the tissue-treatment tip defines a housing that is shaped to define a divider that separates between a first one of the tissue-contact elements and a second one of the tissue-contact elements.

In an application, the divider includes an insulating material.

There is further provided, in accordance with an application of the present invention, apparatus for use in a surgical procedure, the apparatus including:
a tool including:
   a handle at a proximal region of the tool;
   an elongate shaft extending in a distal direction from the handle, the elongate shaft having proximal and distal portions, the distal portion of the shaft being sized and shaped to be inserted into a subject during a surgical procedure;
   a tissue-treatment tip disposed at the distal portion of the shaft; the tissue-treatment tip configured to contact tissue of the subject:
      (a) defining a tissue-treatment tip surface that is configured to face the tissue, and
      (b) being shaped to define a cavity having an internal surface;
   an optical fiber positioned to emit laser energy into the cavity, and:
      a portion of the internal surface is configured to absorb the laser energy and thermally conduct the absorbed energy to the tissue-treatment tip surface, and
      the tissue-treatment tip surface is configured to thermally conduct the absorbed energy to the tissue by contacting the tissue; and
   a motor operatively coupled to the tissue-treatment tip and configured to induce motion of the tissue-treatment tip with respect to the tissue, and the tissue-treatment tip is configured to treat the tissue by the induced motion while the tissue-treatment tip surface is at an elevated temperature due to the thermally conducted absorbed energy.

In an application:
the motor is configured to induce motion of the tissue-treatment tip by transferring a mechanical force to the tissue-treatment tip, and
the tissue-treatment tip is configured to trim a portion of the tissue, by the induced motion, using a mechanical force transferred to the tissue-treatment tip that is lower than a mechanical force transferred to the tissue-treatment tip that would be required to trim the tissue in the absence of the thermally conducted absorbed energy.

In an application, the motor is housed within the handle.

In an application, the motor is configured to induce rotational motion of the tissue-treatment tip with respect to the tissue.

In an application, the motor is configured to induce rotational motion of the tissue-treatment tip with respect to the optical fiber.

In an application, the motor is configured to induce reciprocating motion of the tissue-treatment tip with respect to the tissue.

In an application, the motor is configured to induce longitudinally reciprocating motion of the tissue-treatment tip with respect to the tissue.

In an application, the motor is configured to induce rotationally reciprocating motion of the tissue-treatment tip with respect to the tissue.

In an application, the optical fiber is coupled to a portion of the tissue-treatment tip.

In an application, the optical fiber is coupled to the internal surface of the cavity.

In an application, the optical fiber is fixedly coupled to a portion of the tissue-treatment tip.

In an application, the optical fiber is fixedly coupled to a textured portion of the tissue-treatment tip.

In an application, the optical fiber is fixedly coupled to the portion of the tissue-treatment tip by a saline-impermeable fused silica seal.

There is further provided, in accordance with an application of the present invention, apparatus for use in a surgical procedure, the apparatus including:
a tool including:
   a handle at a proximal region of the tool;
   an elongate shaft extending in a distal direction from the handle, the elongate shaft having proximal and distal portions, the distal portion of the shaft:
      sized and shaped to be inserted into a subject during a surgical procedure, and
      defining a window in a wall of the elongate shaft;
   a beam deflector at the distal portion of the shaft, the beam deflector having a deflector surface;
   a blade disposed at the distal portion of the shaft;
   a motor configured to induce motion of the blade with respect to tissue of a subject; and
   an optical fiber positioned to emit laser energy into the beam deflector, and:
a portion of the deflector surface is configured to reflect the laser energy through the window and to the blade, and
the blade is configured to:
   absorb the reflected laser energy,
   thermally conduct the absorbed energy to the tissue by contacting the tissue, and trim the tissue by the induced motion while the blade is at an elevated temperature due to the absorbed energy.

In an application:
the motor is configured to induce motion of the blade by transferring a mechanical force to the blade, and
the blade is configured to trim the portion of the tissue, by the induced motion, using a mechanical force transferred to the blade that is lower than a mechanical force transferred to the blade that would be required to trim the tissue in the absence of the absorbed energy.

In an application, the motor is housed within the handle.

In an application:
the beam deflector is shaped to define a cavity, and
the portion of the deflector surface that is configured to reflect the laser energy, through the window and to the blade, is an internal surface of the cavity.

In an application, the portion of the internal surface of the cavity is a reflective coating.

In an application, the beam deflector includes an optical light guide.

In an application, the optical light guide includes sapphire or diamond.

In an application, the optical light guide includes a material having a melting point that is higher than 1700 degrees Celsius.

There is further provided, in accordance with an application of the present invention, apparatus for use with tissue of a subject, the apparatus including:
a tool including:
   a handle at a proximal region of the tool;
   an elongate shaft extending in a distal direction from the handle, the elongate shaft having proximal and distal portions, the distal portion of the shaft being sized and shaped to be inserted into a subject during a surgical procedure;
   a tissue-treatment tip disposed at the distal portion of the shaft and defining a tissue-treatment tip surface that is configured to face the tissue of the subject; and
   an optical fiber coupled to the tissue-treatment tip and positioned to emit laser energy into the tissue-treatment tip,
      and the tissue-treatment tip is configured to:
absorb the emitted laser energy,
thermally conduct the absorbed energy from the tissue-treatment tip surface to the tissue, and
treat tissue while the tissue-treatment tip surface is at an elevated temperature due to the absorbed energy.

In an application, the optical fiber is fixedly coupled to a portion of the tissue-treatment tip.

In an application, the optical fiber is fixedly coupled to the tissue-treatment tip by a saline-impermeable fused silica seal.

In an application, the optical fiber is fixedly coupled to the tissue-treatment tip by a saline-impermeable epoxy seal.

In an application, the optical fiber is fixedly coupled to a portion of the tissue-treatment tip that has a textured surface.

In an application, the tissue-treatment tip is shaped to define a cavity, and the optical fiber is coupled to the tissue-treatment tip at the cavity.

In an application:
the cavity has an internal surface,
a first portion of the internal surface is shaped and positioned so as to reflect the laser energy toward the tissue-treatment tip surface, and
a second portion of the internal surface is configured to absorb the laser energy and thermally conduct the absorbed energy to the tissue-treatment tip surface.

In an application, the cavity has a cavity-volume, and the optical fiber occupies a portion of the cavity-volume.

In an application, the cavity has a cavity-volume, and the optical fiber occupies less than 25 percent of the cavity-volume.

In an application:
the tool includes a motor configured to induce motion of the tissue-treatment tip with respect to the tissue, and
the tissue-treatment tip is configured to treat the tissue by the induced motion while the tissue-treatment tip surface is at an elevated temperature due to the absorbed energy.

In an application, the tissue-treatment tip is configured to trim a portion of the tissue, by the induced motion, using a mechanical force transferred to the tissue-treatment tip that is lower than a mechanical force transferred to the tissue-treatment tip that would be required to trim the tissue in the absence of the absorbed energy.

In an application, the motor is housed within the handle.

In an application, the motor is configured to induce rotational motion of the tissue-treatment tip with respect to the tissue.

In an application, the motor is configured to induce rotational motion of the tissue-treatment tip with respect to the optical fiber.

In an application, the motor is configured to induce reciprocating motion of the tissue-treatment tip with respect to the tissue.

In an application, the motor is configured to induce longitudinally reciprocating motion of the tissue-treatment tip with respect to the tissue.

In an application, the motor is configured to induce rotationally reciprocating motion of the tissue-treatment tip with respect to the tissue.

There is further provided, in accordance with an application of the present invention, apparatus for use with tissue of a subject, the apparatus including:
a tool including:
a handle at a proximal region of the tool;
an elongate shaft extending in a distal direction from the handle, the elongate shaft having proximal and distal portions, the distal portion of the shaft being sized and shaped to be inserted into a subject during a surgical procedure;
a tissue-treatment tip protruding from the distal portion of the shaft;
a blade disposed at the distal portion of the shaft;
a motor configured to induce motion of the blade with respect to the tissue; and
an optical fiber and positioned to emit laser energy into the tissue-treatment tip, and the tissue-treatment tip is configured to treat tissue by contacting the tissue while the laser energy is emitted into the tissue-treatment tip.

In an application, the motor is housed within the handle.

In an application:
the tissue-treatment tip is shaped to define a thermal-treatment tip,
an optical fiber is positioned to emit laser energy into the thermal-treatment tip, and
the thermal-treatment tip is configured to treat tissue by contacting the tissue while the laser energy is emitted into the thermal-treatment tip.

In an application, the thermal-treatment tip is configured to coagulate tissue by contacting the tissue while the laser energy is emitted into the thermal-treatment tip.

In an application, the thermal-treatment tip is configured to vaporize tissue by contacting the tissue while the laser energy is emitted into the thermal-treatment tip.

In an application, the optical fiber is coupled to a portion of the tissue-treatment tip.

In an application, the optical fiber is fixedly coupled to a portion of the tissue-treatment tip.

In an application, the optical fiber is fixedly coupled to the portion of the tissue-treatment tip by a saline-impermeable epoxy seal.

In an application, the optical fiber is fixedly coupled to the portion of the tissue-treatment tip by a saline-impermeable fused silica seal.

In an application, the optical fiber is fixedly coupled to a portion of the tissue-treatment tip that has a textured surface.

In an application, the tissue-treatment tip is shaped to define a cavity, and the optical fiber is coupled to the tissue-treatment tip at the cavity.

In an application, the cavity has a cavity-volume, and the optical fiber occupies a portion of the cavity-volume.

In an application, the cavity has a cavity-volume, and the optical fiber occupies less than 25 percent of the cavity-volume.

In an application:
the tool includes:
   an outer cannula:
      on which the tissue-treatment tip is disposed, and
      that defines an outer-cannula lumen; and
   an inner cannula that:
      is disposed within the outer-cannula lumen, and
      is shaped to define the blade,
the motor is configured to induce motion of the inner cannula with respect to the tissue, and
the motion of the inner cannula within the outer-cannula lumen facilitates trimming of tissue that is disposed within the outer-cannula lumen by moving the blade within the outer-cannula lumen.

In an application, the motor is configured to induce rotational motion of the inner cannula with respect to the tissue.

There is further provided, in accordance with an application of the present invention, a method for preparing apparatus, the method including:
positioning a distal end of an optical fiber facing a surface of a metal component; and
delivering laser energy through the optical fiber such that fused silica of the optical fiber melts onto the surface of the metal component, coupling the distal end of the optical fiber to the surface of the metal component.

In an application, the step of positioning includes positioning the distal end of the optical fiber within 0-300 microns of the surface of the metal component.

In an application, the melting point of the metal component is higher than the melting point of the fused silica of the optical fiber.

In an application, the step of delivering includes emitting the laser energy from the distal end, such that fused silica of the optical fiber melts onto the surface, coupling the distal end of the optical fiber to the surface by a saline-impermeable seal of melted fused silica.

In an application:
using the coupled optical fiber and metal component, assembling a surgical device.

In an application:
prior to the step of delivering, roughening a texture of a fiber-binding portion of the surface of the metal component.

In an application, the step of roughening includes increasing a surface area of the fiber-binding portion of the surface of the metal component by at least 10 percent.

In an application, the step of roughening includes increasing the surface area of the fiber-binding portion of the surface of the metal component by at least a factor of two.

In an application, the step of roughening includes, using a laser, etching the surface of the metal component.

In an application, the step of etching includes etching a plurality of pores, each of the pores having a ratio of width to depth of between 1:2 and 1:20.

In an application, the step of etching includes etching a plurality of pores, each of the pores having a ratio of width to depth of between 1:4 and 1:20.

The present invention will be more fully understood from the following detailed description of applications thereof, taken together with the drawings, in which:

### BRIEF DESCRIPTION OF THE DRAWINGS

FIGS. 1, 2A-C and 3A-C are schematic illustrations showing tissue-treatment devices, in accordance with some applications of the invention;

FIGS. 4 and 5A-B are schematic illustrations showing a tissue-treatment device, in accordance with some applications of the invention;

FIG. 6 is a flowchart that schematically illustrates a method for coupling an optical fiber to a metal component, in accordance with some applications of the invention;

FIGS. 7A-D are schematic illustrations showing configurations for coupling an optical fiber to tissue-treatment tips, as well as results of experiments performed using each configuration, in accordance with some applications of the invention;

FIGS. 8-9 are schematic illustrations showing a tissue-treatment device, in accordance with some applications of the invention;

FIGS. 10, 11A-B, 12A-B and 13A-B are schematic illustrations showing a tissue-treatment device, in accordance with some applications of the invention;

FIGS. 14-15, 16A-B and 17A-B are schematic illustrations showing a tissue-treatment device, in accordance with some applications of the invention; and

FIGS. 18A-B, 19A-B and 20A-B are schematic illustrations showing a tissue-treatment device, in accordance with some applications of the invention.

### DETAILED DESCRIPTION OF EMBODIMENTS

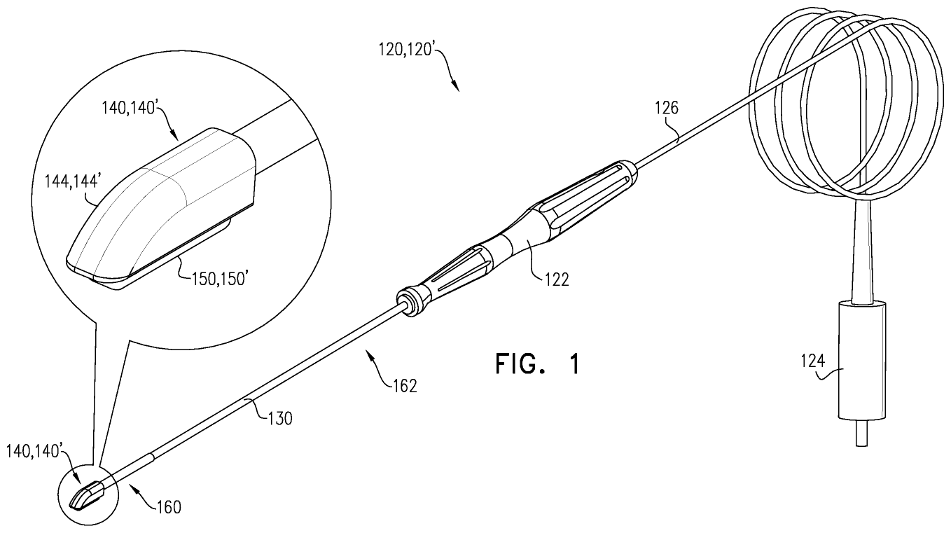

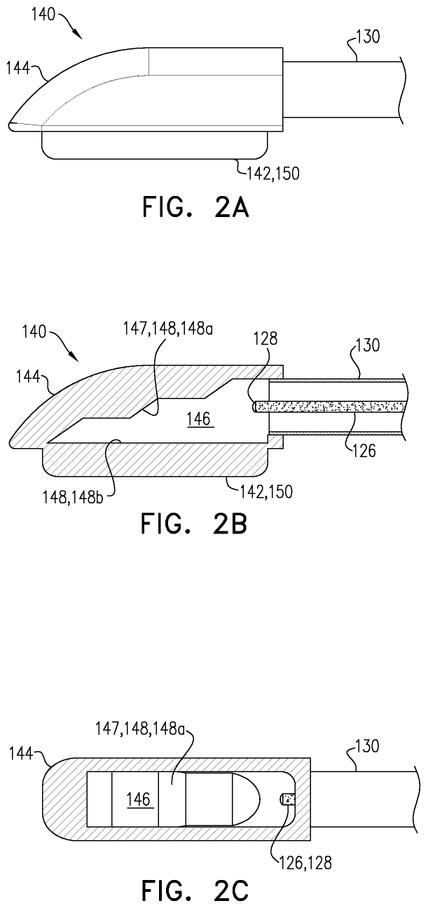

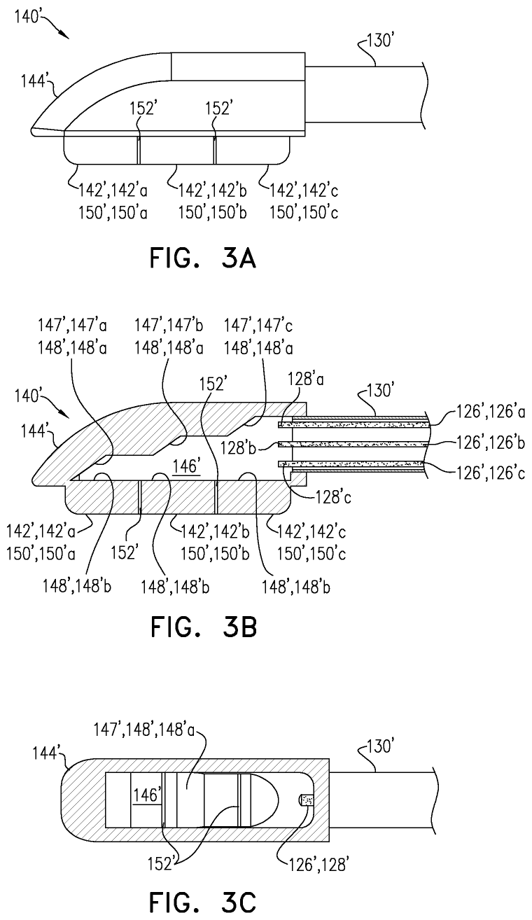

Reference is made to FIGS. 1, 2A-C and 3A-C, which are schematic illustrations showing tissue-treatment devices 120 and 120′, in accordance with some applications of the invention.

Typically, tissue-treatment devices 120, 120′ each are used with or comprise a laser energy source. FIG. 1 shows tissue-treatment devices 120, 120′ comprising a laser energy interface 124 that delivers laser energy from the laser energy source, by an optical fiber 126, through an elongate shaft 130 and toward a tissue-treatment tip 140, 140′ that is disposed at a distal portion 160 of the shaft.

Tissue-treatment devices 120, 120′ are typically used (e.g., by a surgeon) to treat tissue of a subject, and therefore comprise a handle 122 at a proximal region 162 of shaft 130. Handle 122 is typically used to advance tissue-treatment tip 140, 140′ to tissue that is desired to be treated (i.e., a target tissue). Some target tissues include tissue of a joint (e.g., a knee, shoulder, hip or spine) such as a bone, a tendon or cartilaginous tissue such as a meniscus. For some applications, an angular orientation of tissue-treatment tip 140, 140′ is fixed with respect to an angular orientation of handle 122. For some such applications, a distance from handle 122 to tissue-treatment tip 140, 140′ is fixed.

For some applications, and as shown, tissue-treatment tip 140, 140′ has a contoured housing 144, 144′ that is shaped to reduce trauma to surrounding tissue while a tissue-contact element 150 (e.g., a tissue-treatment tip surface 142 thereof) of the tissue-treatment tip contacts the target tissue.

FIGS. 2A-C show a side-view and cross-sections of tissue-treatment tip 140. As shown, optical fiber 126 extends through shaft 130 and into a beam deflector 146 in the interior of tissue-treatment tip 140. For some applications, and as shown, distal end 128 of optical fiber 126 is positioned such that laser energy that is delivered from laser energy interface 124, along optical fiber 126, is emitted from distal end 128 of optical fiber 126 and enters beam deflector 146.

When laser energy is emitted from optical fiber 126, a deflector surface 148 of beam deflector 146 directs the laser energy toward tissue-treatment tip surface 142 of tissue-contact element 150. For this purpose, deflector surface 148 has a first portion 148a that is shaped and positioned to reflect the laser energy toward a second portion 148b of the deflector surface 148. For some applications, first portion 148, 148a is a reflective coating, e.g., comprising gold and/or silver. For some applications, first portion 148a of deflector surface 148 includes one or more angled surfaces 147, as shown in FIGS. 2B and 2C.

For some applications, beam deflector 146 defines a cavity, such that deflector surface 148 defines the internal surface of the cavity. For some such applications, first portion 148a and second portion 148b of deflector surface 148 each define opposing internal surfaces of the cavity.

For some applications, beam deflector 146 comprises an optical light guide. Typically for such applications, deflector surface 148 defines the surface of the optical light guide. Further typically for such applications, first portion 148a of deflector surface 148 has at least one reflective surface 147 that is disposed at an angle so as to reflect the laser energy from optical fiber 126 toward second portion 148b of deflector surface 148, e.g., utilizing total internal reflection.

For some applications in which beam deflector 146 comprises an optical light guide, the optical light guide comprises material having a high melting point (e.g., a melting point higher than 1700 degrees Celsius), such as sapphire or diamond.

For some applications, and as shown, second portion 148b of the deflector surface 148 is defined by or is adjacent to tissue-contact element 150. Typically for such applications, laser energy that is absorbed by second portion 148b undergoes photothermal conversion and is thermally conducted to tissue-treatment tip surface 142. It is therefore typically desirable that tissue-contact element 150, which defines second portion 148b, conduct heat efficiently. Suitable materials for tissue-contact element 150 include, for example, tungsten, molybdenum, stainless steel, cobalt, chrome and/or alloys thereof.

For some applications, the tissue-treatment tip, e.g., tissue-treatment tip 140′ (of tissue-treatment device 120′) shown in FIGS. 3B-C, has more than one optical fiber 126. For example, tissue-treatment tip 140′ may have three optical fibers 126a, 126b, 126c as shown in FIG. 3B. For some applications, the use of more than one optical fiber facilitates regulation of an amplitude of laser energy that is transmitted to a beam deflector 146′, e.g., by selecting through how many of the optical fibers to deliver laser energy. Typically for such applications, tissue-treatment device 120′ comprises a controller (e.g., comprising circuitry on handle 122) that is configured to separately control emission of laser energy from each optical fiber.

Tissue-treatment tip 140′ is generally otherwise similar in structure and function to tissue-treatment tip 140 described hereinabove. Components bearing corresponding reference numerals (e.g., beam deflector 146 and beam deflector 146′) are typically interchangeable between the respective tissue-treatment tips. The following description will therefore focus on features that are particular to tissue-treatment tip 140′.

For some applications, each optical fiber 126′ is positioned to emit laser energy into a respective region of beam deflector 146′. As described hereinbelow, emitting laser energy into a selected region of beam deflector 146′ causes selective heating of a given portion of tissue-contact element 150′, and therefore conduction of heat to a selected portion of tissue that is in contact with the tissue-contact element.

For some such applications, and as shown, each distal end 128′a, 128′b, 128′c of respective optical fibers 126′a, 126′b, 126′c faces a respective angled surface 147′a, 147′b, 147′c of deflector surface 148′. Emitting laser energy from a given optical fiber 126′ therefore causes the laser energy to be emitted toward a respective region (e.g., a respective angled surface 147) of a first portion 148′a of deflector surface 148′. Each region of a second portion 148′b of deflector surface 148′ in turn absorbs the laser energy and thermally conducts the absorbed energy to a respective portion of tissue-treatment tip surface 142 (e.g., tissue-treatment tip surfaces 142′a, 142′b, 142′c of tissue-contact element 150′a, 150′b and 150′c, as shown in FIGS. 3A-B).

For some applications, and as shown in FIG. 3C, housing 144′ of tissue-treatment tip 140′ is shaped to define dividers 152 that separate between discrete tissue-contact elements 150′a, 150′b and 150′c, inhibiting conduction of heat between tissue-contact elements and thereby further facilitating selective heating of the tissue-contact elements. For some applications, dividers 152 comprise an insulating material (e.g., a ceramic material, such as zirconium dioxide) that inhibits conduction of heat between tissue-contact elements 150′a, 150′b and 150′c. Alternatively or in addition, dividers 152 may be empty spaces that inhibit conduction of heat between the respective tissue-contact elements.

Thus, at least a portion of tissue-contact element 150, 150′ is brought into contact with target tissue and heated (not necessarily in that order). For some applications, contacting the target tissue with tissue-treatment tip 140, 140′ results in vaporization of at least some of the target tissue. Alternatively or in addition, contacting the target tissue with tissue-treatment tip 140, 140′ results in coagulation of at least some of the target tissue.

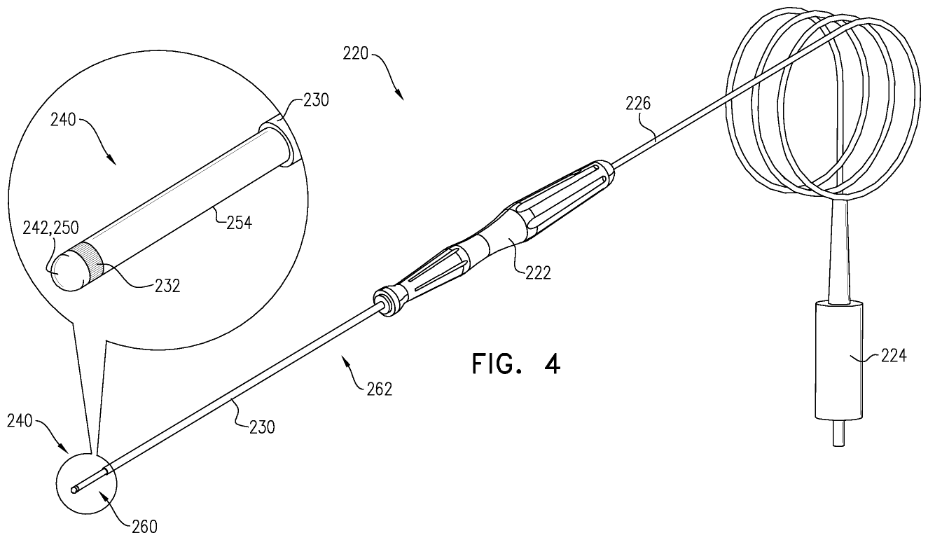

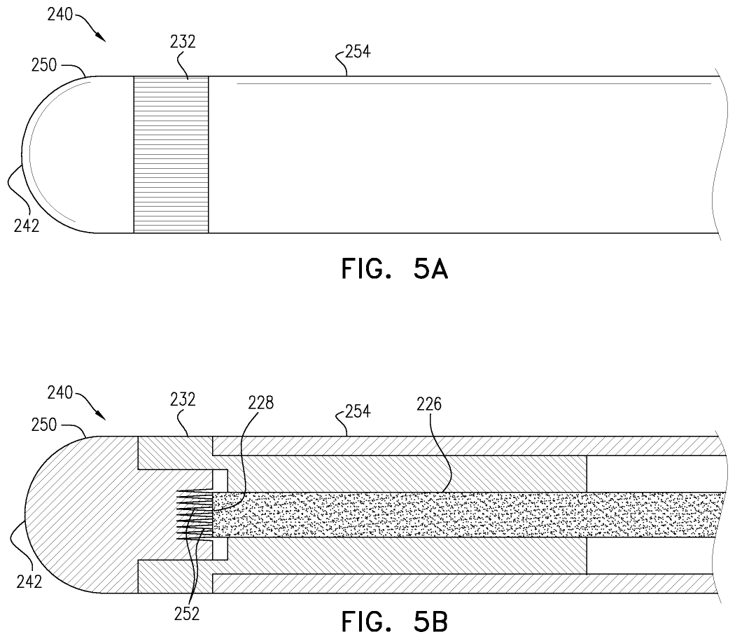

Reference is made to FIGS. 4 and 5A-B, which are schematic illustrations showing a tissue-treatment device 220, in accordance with some applications of the invention.

Similarly to tissue-treatment device 120, tissue-treatment device 220 is used to treat tissue while a tissue-treatment tip surface 242 of tissue-treatment tip 240 is at an elevated temperature due to laser energy that the tissue-treatment tip absorbs.

Typically, tissue-treatment device 220 is used with or comprises a laser energy source. FIG. 4 shows tissue-treatment device 220 comprising a laser energy interface 224 that delivers laser energy from the laser energy source, by an optical fiber 226, through an elongate shaft 230 and toward tissue-treatment tip 240 that is disposed at a distal portion 260 of the shaft. Tissue-treatment device 220 is also typically used (e.g., by a surgeon) to treat tissue of a subject, and therefore comprises a handle 222 at a proximal region 262 of shaft 230. Handle 222 is typically used to advance tissue-treatment tip 240 to the target tissue. Some target tissues include tissue of a joint (e.g., a knee, shoulder, hip or spine) such as a bone, a tendon or cartilaginous tissue such as a meniscus. For some applications, an angular orientation of tissue-treatment tip 240 is fixed with respect to an angular orientation of handle 222. For some such applications, a distance from handle 222 to tissue-treatment tip 240 is fixed.

Further similarly to tissue-treatment tip surface 142 of tissue-treatment tip 140, the tissue-treatment tip absorbs laser energy that is emitted from optical fiber 226, and when tissue-treatment tip surface 242 is placed in contact with the target tissue, the tissue-treatment tip surface thermally conducts the absorbed energy to the tissue. Typically, tissue-treatment tip 240 conducts energy to the target tissue.

FIGS. 5A-B are side and cross-sectional views of tissue-treatment tip 240 showing positioning of optical fiber 226 in relation to tissue-treatment tip 240 (e.g., to a tissue-contact element 250 thereof) in a manner that facilitates emission of laser energy from the optical fiber (e.g., from distal end 228 thereof) and into the tissue-treatment tip.

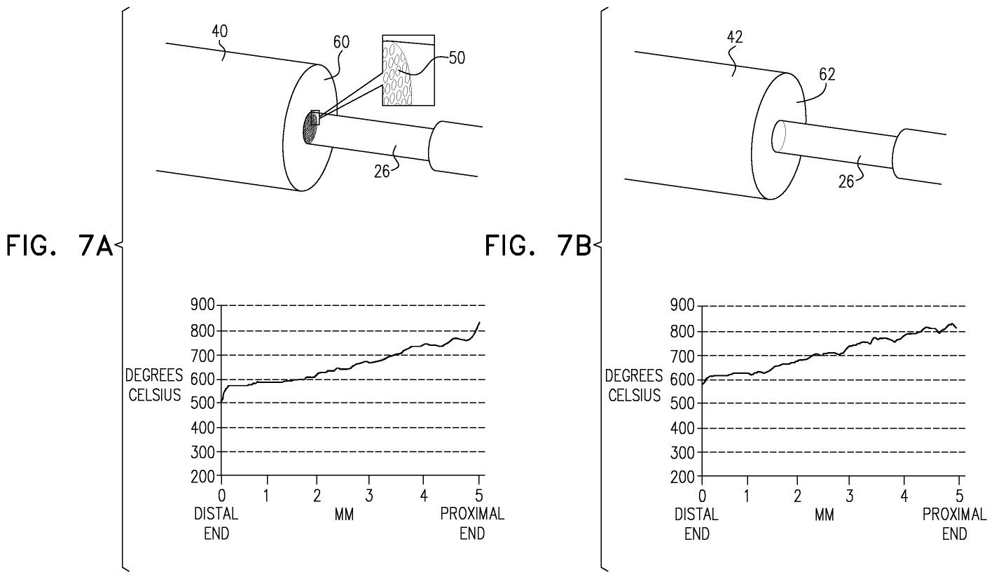

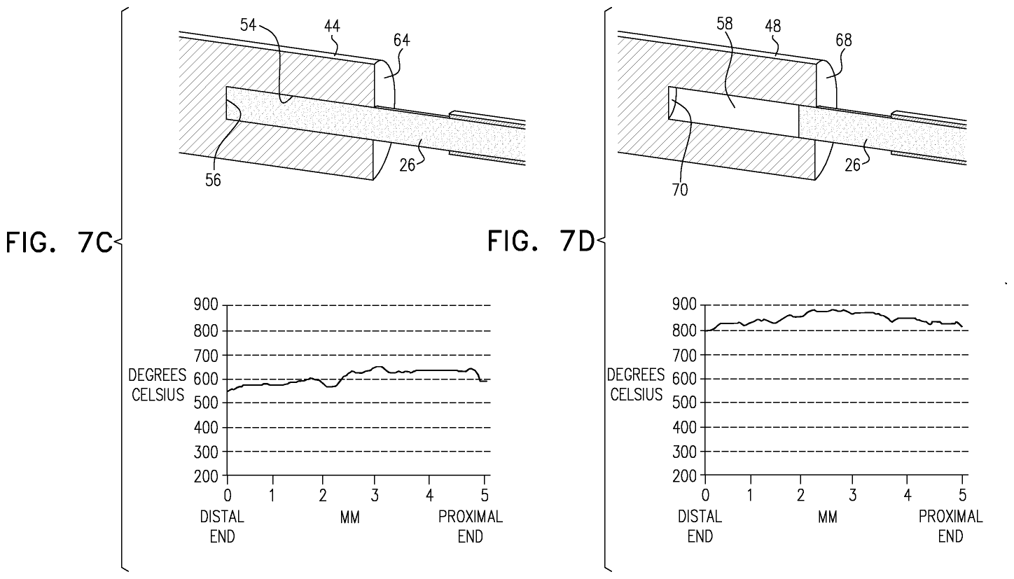

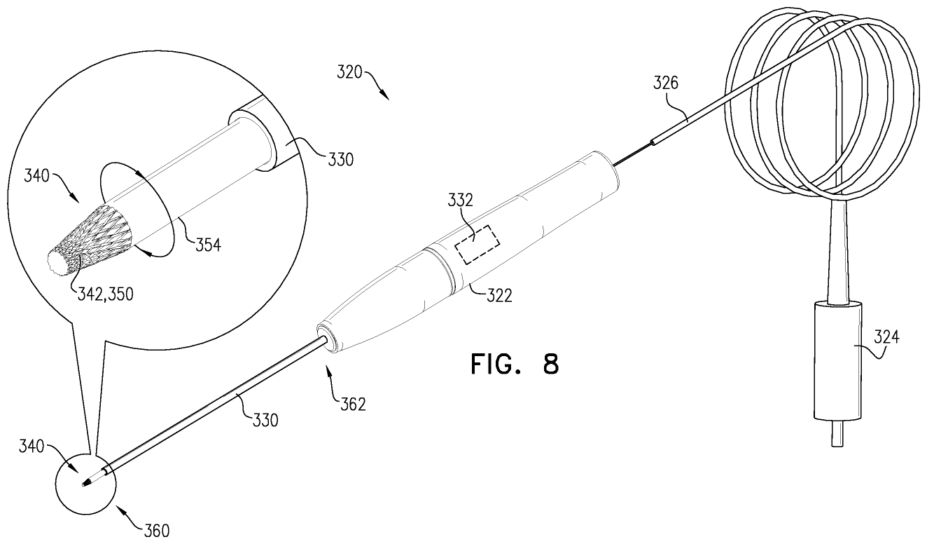

For example, and as shown, distal end 228 of optical fiber 226 may be positioned facing (e.g., near or contacting) a plurality of pores 252 that are etched into a surface of contact element 250. The presence of pores 252 increases a surface area of the portion of the surface of contact element 250 that faces distal end 228 of optical fiber 226. This exemplary embodiment of pores 252 is also shown below in FIG. 7A, and other non-limiting examples showing positioning of optical fiber 226 in relation to tissue-treatment tip 240 are presented hereinbelow in FIGS. 7B-D.

It is hypothesized by the inventors that the increased surface area of the portion of the surface of contact element 250 improves efficiency of photothermal conversion, thereby enabling heating the contact element to higher temperatures without increasing the laser energy, while protecting the laser source by limiting backscattering of the laser energy.

For some applications, and as shown, tissue-treatment tip 240 comprises an isolator 232 (e.g., a ceramic isolator) that insulates tissue-contact element 250 from other elements of tissue-treatment device 220, such as tip cannula 254.

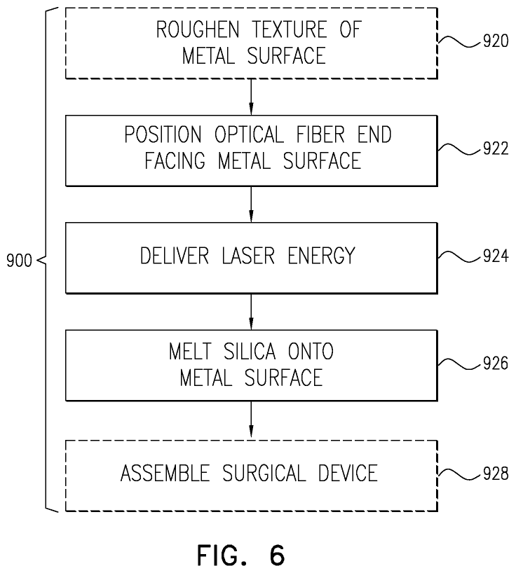

Reference is made to FIG. 6, which is a flowchart that schematically illustrates a method 900 for coupling an optical fiber to a metal component in accordance with some applications of the invention, and to FIGS. 7A-D, which are schematic illustrations showing configurations for coupling an optical fiber 26 to tissue-treatment tips 40, 42, 44 and 48, as well as results of experiments performed using each configuration, in accordance with some applications of the invention.

For some applications, prior to coupling the optical fiber to the metal surface, the metal surface (e.g., a fiber-binding portion thereof) is first roughened (step 920). For some such applications, roughening increases a surface area of the fiber-binding portion by at least 10 percent. For example, roughening may increase the surface area of the fiber-binding portion by at least a factor of two.

For some such applications, the surface is roughened by using a laser to etch the fiber-binding portion of the metal surface. For example, the laser may be used to etch a plurality of pores, each of the pores having a ratio of width to depth of between 1:2 and 1:20, e.g., of between 1:4 and 1:20.

In experiments conducted by the inventors, a 2.4 mm diameter portion of the surface of a Tungsten-Molybdenum alloy was roughened by etching using a 1064 nm wavelength laser operating at 15 Watts. Each pore position was illuminated with the laser for at least 100 pulses of 40 nanoseconds each in duration, which etched generally conical pores (shown schematically in FIGS. 5B and 7A) having a diameter of 10-50 microns and a depth of 100-600 microns, at a distance of 15-100 microns from pore-center to pore-center.

Alternatively or in addition, the fiber-binding portion may be roughened using mechanical pressure and/or chemical etching.

In step 922, a distal end of the optical fiber is positioned facing the surface (e.g., within 0-300 microns of the surface) of the metal component.

While the distal end of the optical fiber is facing the surface, laser energy is delivered through the optical fiber (step 924). In one of the experiments conducted by the inventors, 20 W of 980 nm laser energy, was emitted from a 400 micron diameter optical fiber to a cylindrical (1.6 mm by 5 mm) tissue-treatment tip, for 3-8 seconds. All experiments described herein were conducted at room temperature

The laser energy is emitted from the distal end of the optical fiber to the surface of the metal component, elevating the temperature of: (i) fused silica of the optical fiber and (ii) the metal component. For some applications, the melting point of the metal component is higher than the melting point of the fused silica, such that the distal end and the metal component may be heated to a temperature at which the fused silica melts, yet the metal surface does not melt.

Typically, the elevated temperature of the optical fiber causes fused silica of the optical fiber to melt onto the surface of the metal component (step 926). Melting the fused silica onto the surface typically couples the distal end of the optical fiber to the surface. For some applications, the fused silica is melted onto the surface so as to create a seal of fused silica that is impermeable to body fluids and saline or other liquids used in a surgical procedure. It is hypothesized by the inventors that the seal of fused silica also adds to the mechanical strength of the connection between the optical fiber and the surface of the metal component.

For some applications, the coupled optical fiber and metal component are then used to assemble a surgical device (step 928), such as tissue-treatment devices described herein with reference to: FIGS. 4 and 5A-B; 10 and 11A-B; 14-15, 16A-B and 17A-B; and 18A-B, 19A-B and 20A-B.

FIGS. 7A-D show four configurations for coupling optical fiber 26 to a tissue-treatment tip 40, 42, 44, 48 (e.g., at a surface of the tissue-treatment tip or at a cavity defined by the tissue-treatment tip), as well as temperature measurements for tissue-treatment tips heated using each configuration.

For each configuration, 35 W of 980 nm laser energy was emitted from a 400 micron-diameter optical fiber to a free-standing cylindrical (1.6 mm by 5 mm) tissue-treatment tip for 1 second. A thermal camera was used to measure the temperature along the length of the tissue-treatment tip 1 second after beginning the emission of the laser energy. Each configuration of an optical fiber coupled to a tissue-treatment tip was prepared generally according to method 900 described hereinabove, as well as in accordance with additional parameters described hereinbelow that differentiate between each configuration.

The upper pane of FIG. 7A shows optical fiber 26 coupled to a surface 60 of a tissue-treatment tip 40 that had been roughened to define pores 52 according to step 920 of method 900 described hereinabove. The graph in the lower pane of FIG. 7A shows that the temperature of the tissue-treatment tip 40 ranged between 500 and 900 degrees Celsius along a length of the tissue-treatment tip.

The upper pane of FIG. 7B shows optical fiber 26 coupled to surface 62 of a tissue-treatment tip 42 that had not been roughened to define pores. The graph in the lower pane of FIG. 7B shows that the temperature of the tissue-treatment tip 42 also ranged between 500 and 900 degrees Celsius along a length of the tissue-treatment tip.

For some applications in which the tissue-treatment tip defines a cavity, and as shown in FIGS. 7C-D, optical fiber 26 is coupled at the cavity such that the optical fiber occupies a portion of the cavity.

The upper pane of FIG. 7C shows optical fiber 26 coupled to a tissue-treatment tip 44 that is shaped to define a cavity 54 that is 0.45 mm in diameter and 2 mm deep from a proximal surface 64 of the tissue-treatment tip. Optical fiber 26 was coupled to tissue-treatment tip 44 at a floor 56 of cavity 54. The graph in the lower pane of FIG. 7C shows that the temperature of the tissue-treatment tip 44 ranged between 500 and 700 degrees Celsius along the length of the tissue-treatment tip, which is a temperature range that is (i) lower and (ii) more uniform along a length of the tissue-treatment tip, in comparison to the temperature ranges of the tissue-treatment tips that were coupled to optical fiber 26 at surface 60, 62.

The upper pane of FIG. 7D shows optical fiber 26 coupled to a tissue-treatment tip 48 that is shaped to define a somewhat deeper cavity 58 that is 0.45 mm in diameter and 2.5 mm deep from a proximal surface 68 of the tissue-treatment tip. As shown, optical fiber 26 is coupled to tissue-treatment tip at cavity 58, e.g., to an interior wall of the cavity. Optical fiber 26 was inserted about 0.2 mm below proximal surface 68 of the cavity, at a distance from a floor 70 of cavity 58, such that the optical fiber occupies less than 25 percent of a volume of the cavity. The graph in the lower pane of FIG. 7D shows that the temperature of the tissue-treatment tip 48 ranged between 800 and 900 degrees Celsius along the length of the tissue-treatment tip, which is a temperature range that is (i) higher and (ii) more uniform along the length of the tissue-treatment tip, in comparison to the temperature ranges of the other tissue-treatment tips described above.

These experimental results demonstrate that coupling optical fiber 26 to tissue-treatment tip 48 that is shaped to define cavity 58 (FIG. 7D) has advantages over the configurations shown in FIGS. 7A-C. Notwithstanding the advantages of the features shown in FIG. 7D, the configurations shown in FIGS. 7A-C produce suitable results and are within the scope of the present invention.

Firstly, the internal surface of cavity 58 provides (due to its length) a larger surface area for the interface between the laser energy and the tissue-treatment tip, in comparison to the surface area of surfaces 60, 62 of tissue-treatment tips 40, 42 shown in FIGS. 7A-B. Furthermore, FIG. 7D shows optical fiber 26 coupled to tissue-treatment tip 44 at a distance of about 2.3 mm from floor 70 of cavity 58 that is only 0.2 mm less than a depth of the cavity. This configuration increased the effective surface area of the cavity available for photothermal conversion of the laser energy to heat, in comparison to the configuration shown in FIG. 7C, in which optical fiber 26 reaches floor 56 of cavity 54.

Furthermore, inserting optical fiber 26 0.2 mm into cavity 58 and melting fused silica of the optical fiber (step 926 of method 900 described hereinabove) onto the interior surface of cavity 58 appears to add to the mechanical strength of the connection between the optical fiber and the tissue-treatment tip, in comparison to the configurations shown in FIGS. 7A-B, in which optical fiber 26 is coupled to surface 60, 62 of tissue-treatment tip 40, 42.

It is further hypothesized by the inventors that coupling optical fiber 26 to tissue-treatment tip 48 at a greater distance from floor 70 of cavity 58 protects the laser source by reducing backscattering of the laser energy.

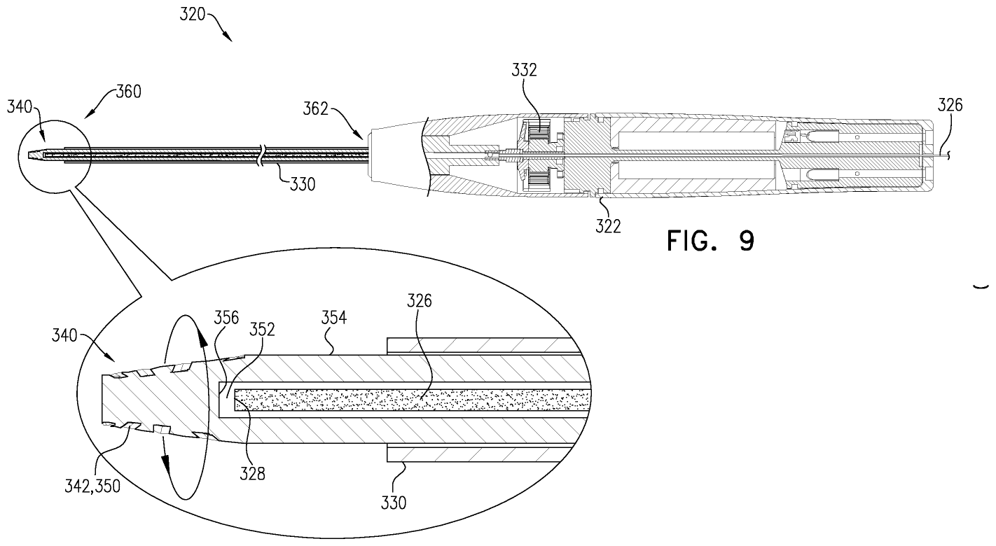

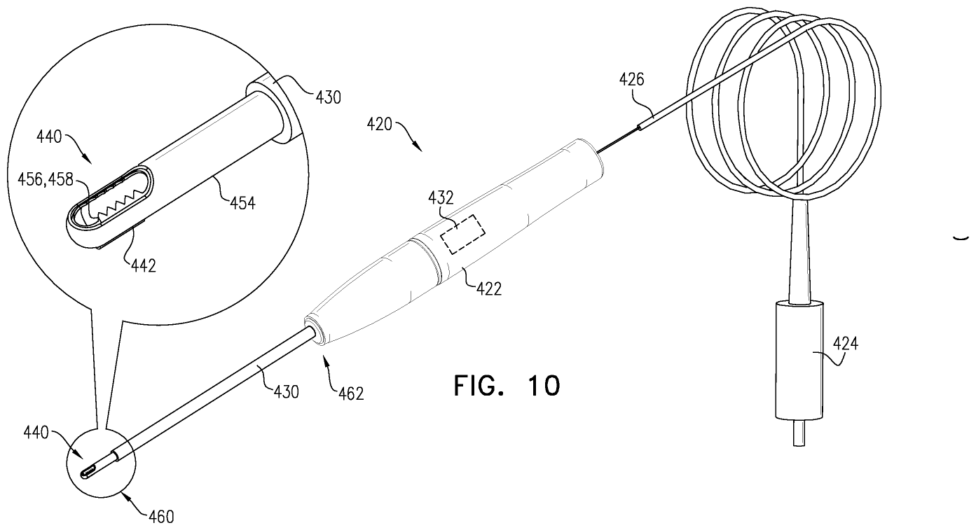

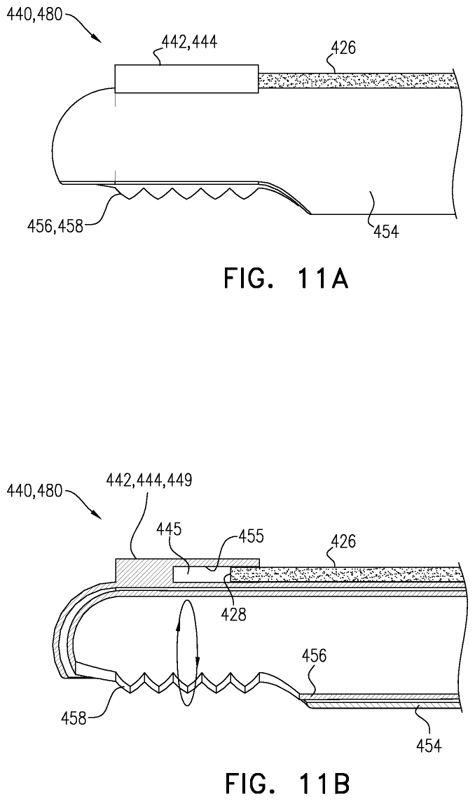

Reference is made to FIGS. 8-9, which are schematic illustrations showing a tissue-treatment device 320, in accordance with some applications of the invention.

As described hereinbelow, tissue-treatment device 320 comprises a tissue-treatment tip 340 that: (i) conducts heat to the target tissue, and (ii) is operatively coupled to a motor 332 that transfers a mechanical force to the tissue-treatment tip, thereby inducing motion of the tissue-treatment tip. In this way, tissue-treatment device 320 makes use of both thermal energy and mechanical force to treat tissue.

Typically, tissue-treatment device 320 is used with or comprises a laser energy source. FIG. 8 shows tissue-treatment device 320 comprising a laser energy interface 324 that delivers laser energy from the laser energy source, by an optical fiber 326, through an elongate shaft 330 and toward tissue-treatment tip 340 that is disposed at a distal portion 360 of the shaft. Tissue-treatment device 320 is also typically used (e.g., by a surgeon) to treat tissue of a subject, and therefore comprises a handle 322 at a proximal region 362 of shaft 330. Handle 322 is typically used to advance tissue-treatment tip 340 to the target tissue). Some target tissues include tissue of a joint (e.g., a knee, shoulder, hip or spine) such as a bone, a tendon or cartilaginous tissue such as a meniscus. For some such applications, a distance from handle 322 to tissue-treatment tip 340 is fixed.

For some applications, and as shown, motor 332 is housed within handle 322. Motor 332 may be operatively coupled to tissue-treatment tip 340 from an alternate location on tissue-treatment device 320 (e.g., the motor may be mounted on shaft 330).

For some applications, and as shown in FIG. 9, motor 332 causes tissue-treatment tip 340 to rotate with respect to the target tissue. For some such applications, motor 332 causes tissue-treatment tip 340 to rotate with respect to optical fiber 326 (e.g., distal end 328 thereof) and/or with respect to shaft 330.

As shown in FIG. 9, tissue-treatment tip 340 is shaped to define a cavity 352 having an internal surface 356, and optical fiber 326 is positioned to emit laser energy into the cavity. At least a portion of internal surface 356 absorbs the laser energy and thermally conducts the absorbed energy to a tissue-treatment tip surface 342 (e.g., of tissue-contact element 350, as shown), thereby elevating the temperature of the tissue-treatment tip surface. In this way, tissue-treatment tip 340 (i) absorbs laser energy that is emitted from optical fiber 326, and (ii) thermally conducts the absorbed energy to the tissue when tissue-treatment tip surface 342 is placed in contact with the target tissue.

For some applications, the use of both absorbed thermal energy and mechanical force reduces an amount of mechanical force that is required for tissue-treatment device 320 to treat (e.g., to trim a portion of) the tissue. For example, even in the absence of the thermal energy, treatment tip 340 may trim the tissue to a certain extent if tissue-contact element 350 rotates while tissue-treatment tip surface 342 contacts the tissue. However, tissue-treatment device 320 may require a greater mechanical force to trim the tissue without the thermal energy. Thus, the thermal energy facilitates trimming the tissue using a lower mechanical force.

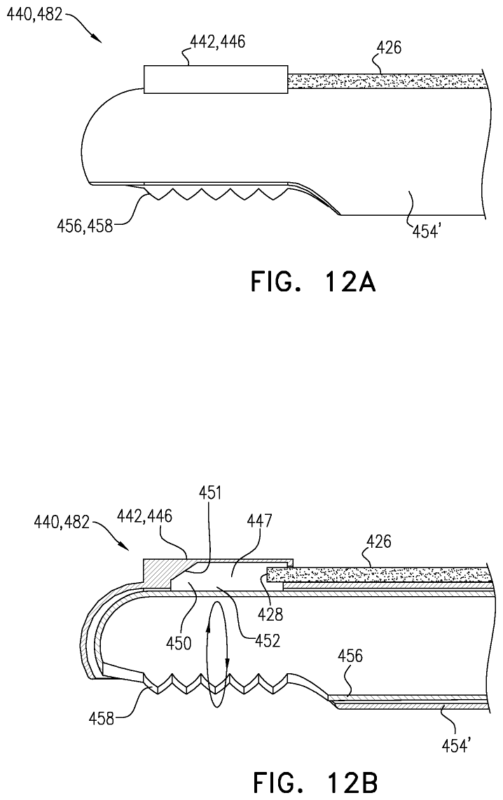

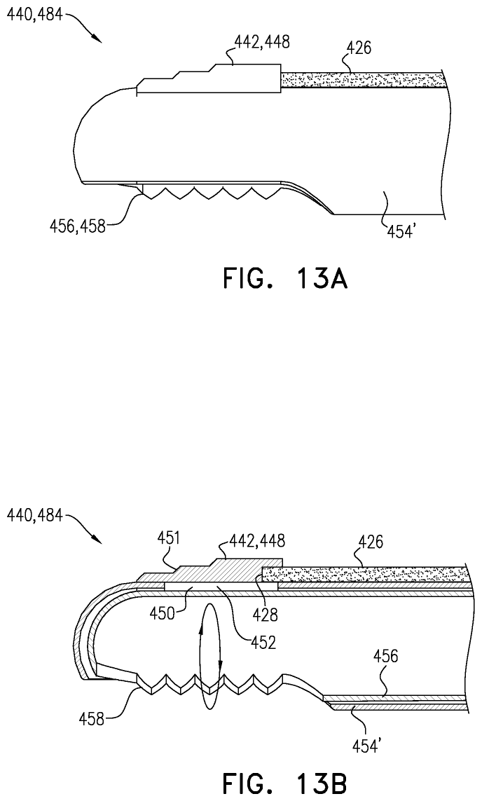

Reference is made to FIGS. 10, 11A-B, 12A-B and 13A-B, which are schematic illustrations showing a tissue-treatment device 420, in accordance with some applications of the invention.

As described hereinbelow, tissue-treatment device 420 comprises a tissue-treatment tip 440 that: (i) conducts heat to the target tissue, and (ii) is operatively coupled to a motor 432 (e.g., similar to motor 332 shown in FIGS. 8-9) that transfers a mechanical force to the tissue-treatment tip, thereby inducing motion of the tissue-treatment tip. In this way, tissue-treatment device 420 makes use of both thermal energy and mechanical force to treat tissue.

Typically, tissue-treatment device 420 is used with or comprises a laser energy source. FIG. 10 shows tissue-treatment device 420 comprising a laser energy interface 424 that delivers laser energy from the laser energy source, by an optical fiber 426, through an elongate sheath 430 and toward tissue-treatment tip 440 that is disposed at a distal portion 460 of the sheath. Tissue-treatment device 420 is also typically used (e.g., by a surgeon) to treat tissue of a subject, and therefore comprises a handle 422 at a proximal region 462 of sheath 430. Handle 422 is typically used to advance tissue-treatment tip 440 to the target tissue. Some target tissues include tissue of a joint (e.g., a knee, shoulder, hip or spine) such as a bone, a tendon or cartilaginous tissue such as a meniscus. For some such applications, a distance from handle 422 to tissue-treatment tip 440 is fixed.

For some applications, and as shown, motor 432 is housed within handle 422. Motor 432 may be operatively coupled to tissue-treatment tip 440 from an alternate location on tissue-treatment device 420 (e.g., the motor may be mounted on sheath 430).

For some applications, and as shown in FIG. 10, tissue-treatment tip 440 comprises a shaft, e.g., an outer cannula 454, 454′ that defines an outer-cannula lumen. Typically for such applications, tissue-treatment tip also comprises an inner cannula 456 that is disposed within the outer-cannula lumen. Further typically for such applications, and as shown, inner cannula 456 is shaped to define a blade 458.

For some applications, and as shown in FIGS. 11A-B, 12A-B and 13A-B, motor 432 causes inner cannula 456, and therefore blade 458 to move (e.g., to rotate, as shown) with respect to the target tissue and/or with respect to optical fiber 426. Typically for such applications, tissue-treatment device 420 is advanced toward target tissue such that the target tissue enters the outer-cannula lumen, and movement of blade 458 within the outer-cannula lumen trims the target tissue.

As described hereinabove, optical fiber 426 is used to deliver laser energy toward tissue-treatment tip 440. For some applications, optical fiber 426 is coupled, e.g., fixedly coupled, to tissue-treatment tip 440. For example, optical fiber 426 may be coupled to laser energy-receiving portion 442 by creating a seal of fused silica (e.g., between optical fiber 426 and laser energy-receiving portion 442) that is impermeable to body fluids and saline or other liquids, as described hereinabove with reference to step 926 of method 900. For example, optical fiber 426 may be coupled to a portion of laser energy-receiving portion 442 having a textured surface. Alternatively or in addition, optical fiber 426 may be coupled to laser energy-receiving portion 442 by an epoxy seal that is impermeable to body fluids and saline or other liquids used in a surgical procedure.

As described hereinabove, optical fiber 426 is positioned to emit laser energy into tissue-treatment tip 440. Typically, the laser energy is photothermally converted into thermal energy that is used to treat the target tissue while the laser energy is emitted into the tissue-treatment tip 440 (e.g., into laser energy-receiving portion 442 thereof). FIGS. 11A-B, 12A-B and 13A-B show three different configurations of laser energy-receiving portion 442.

FIGS. 11A-B show a tissue-treatment tip 440, 480 having a laser energy-receiving portion 442 that defines a thermal-treatment tip 444 that receives emitted laser energy (e.g., from distal end 428 of optical fiber 426) and photothermally converts the laser energy into thermal energy. For some applications, and as shown in FIG. 11B, energy-receiving portion 442 is shaped to define a cavity 445, and optical fiber 426 is coupled to tissue-treatment tip 440, 480 at the cavity. For example, and as shown, optical fiber 426 may occupy a portion (e.g., less than 25 percent) of a volume of cavity 445.

Typically, at least a portion of internal surface 455 of cavity 445 absorbs the emitted laser energy and thermally conducts the absorbed energy to a tissue-facing surface 449 of thermal-treatment tip 444, thereby elevating the temperature of the tissue-facing surface.

In this way, tissue-treatment tip 440, 480 (i) absorbs laser energy that is emitted from optical fiber 426, and (ii) thermally conducts the absorbed energy to the tissue when thermal-treatment tip 444 (e.g., tissue-facing surface 449 thereof) is placed in contact with the target tissue, treating the target tissue. For example, thermal-treatment tip 444 may coagulate and/or vaporize target tissue that the thermal-treatment tip contacts while the laser energy is emitted into tissue-treatment tip 440, 480.

FIGS. 12A-B and 13A-B show alternate configurations of tissue-treatment tip 440, 482, 484, in which laser energy-receiving portion 442 defines beam deflector 446, 448 having a deflector surface 451 that reflects the laser energy toward blade 458.

Typically for such applications, outer cannula 454′ is shaped to define a window 452 through which the laser energy passes, from deflector surface 451 to blade 458. Further typically, blade 458 absorbs the reflected laser energy and is at an elevated temperature due to the absorbed thermal energy while contacting the tissue. In this way, blade 258 thermally conducts the absorbed energy to the tissue by contacting the tissue, which facilitates treating the tissue.

For some applications, the use of both absorbed thermal energy and mechanical force reduces an amount of mechanical force that is required for tissue-treatment tips 440, 482, 484 to treat (e.g., to trim a portion of) the tissue. For example, even in the absence of the thermal energy, rotation of blade 458 while the blade contacts the tissue may trim the tissue to a certain extent. However, tissue-treatment device 420 may require a greater mechanical force to trim the tissue without the thermal energy. Thus, the thermal energy facilitates trimming the tissue using a lower mechanical force.

For some applications, and as shown in FIGS. 12A-B, beam deflector 446 is shaped to define a cavity 447. Typically for such applications, an internal surface of cavity 447 defines deflector surface 451 that reflects the laser energy through window 452 and to blade 458. For some such applications, deflector surface 451 is a reflective coating.

For some applications, and as shown in FIGS. 13A-B, beam deflector 448 defines an optical light guide 448, e.g., comprising sapphire or diamond. For some such applications, optical light guide 448 comprises a material having a melting point that is higher than 1700 degrees Celsius.

Reference is made to FIGS. 14-15, 16A-B and 17A-B, which are schematic illustrations showing a tissue-treatment device 520, 520′, in accordance with some applications of the invention.

As described hereinbelow, tissue-treatment device 520, 520′ comprises a tissue-treatment tip 540, 540′ that: (i) conducts heat to the target tissue, and (ii) is operatively coupled to a motor 534 that transfers a mechanical force to the tissue-treatment tip, thereby inducing motion of the tissue-treatment tip. In this way, tissue-treatment device 520, 520′ makes use of both thermal energy and mechanical force to treat tissue.

Typically, tissue-treatment devices 520, 520′ each are used with or comprise a laser energy source. FIG. 14 shows tissue-treatment devices 520, 520′ comprising a laser energy interface 524 that delivers laser energy from the laser energy source, by an optical fiber 526, through an elongate shaft 530 and toward tissue-treatment tip 540, 540′ that is disposed at a distal portion 560 of the shaft. Tissue-treatment device 520, 520′ is also typically used (e.g., by a surgeon) to treat tissue of a subject, and therefore comprises a handle 522 at a proximal region 562 of shaft 530. Handle 522 is typically used to advance tissue-treatment tip 540, 540′ to the target tissue. Some target tissues include tissue of a joint (e.g., a knee, shoulder, hip or spine) such as a bone, a tendon or cartilaginous tissue such as a meniscus. For some applications, an angular orientation of tissue-treatment tip 540, 540′ is fixed with respect to an angular orientation of handle 522.

For some applications, and as shown, motor 534 is housed within handle 322. For some such applications, motor 534 is operatively coupled to tissue-treatment tip 540, 540′ via a cannula 554, 554′ that is disposed within shaft 530. Motor 534 may be operatively coupled to tissue-treatment tip 540, 540′ from an alternate location on tissue-treatment device 520, 520′ (e.g., the motor may be mounted on shaft 530).

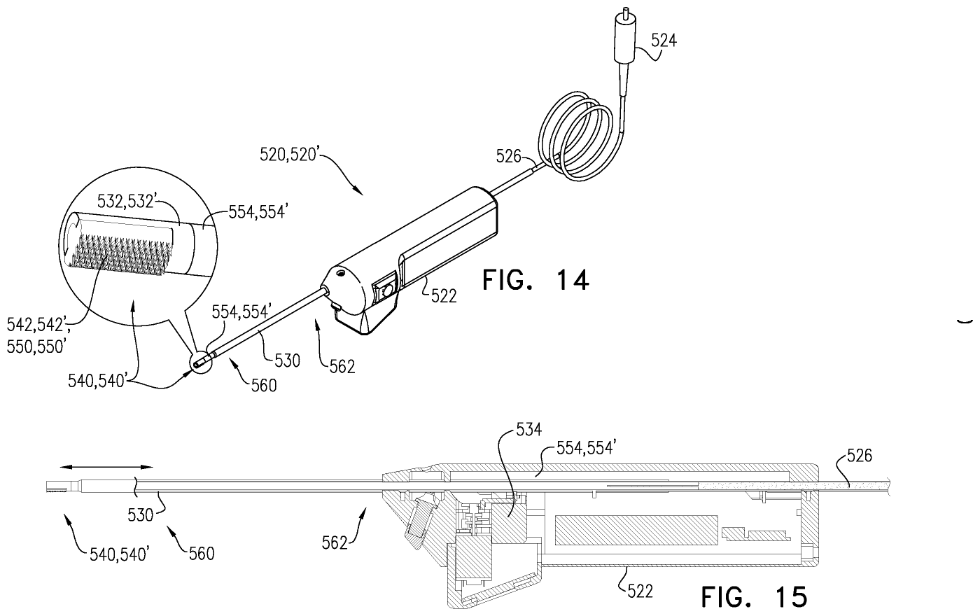

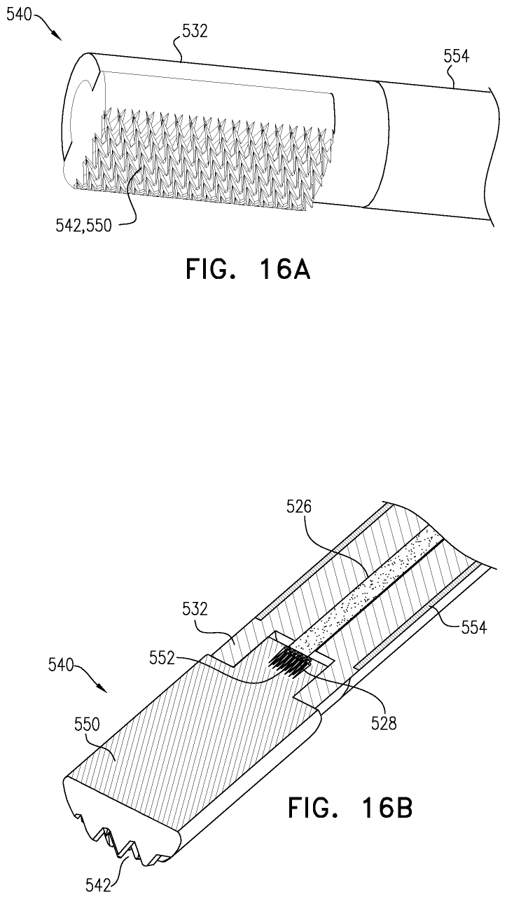

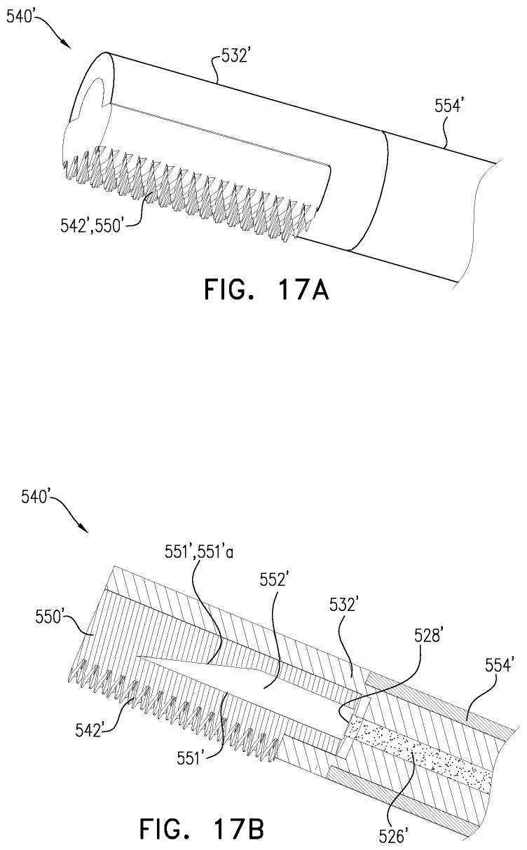

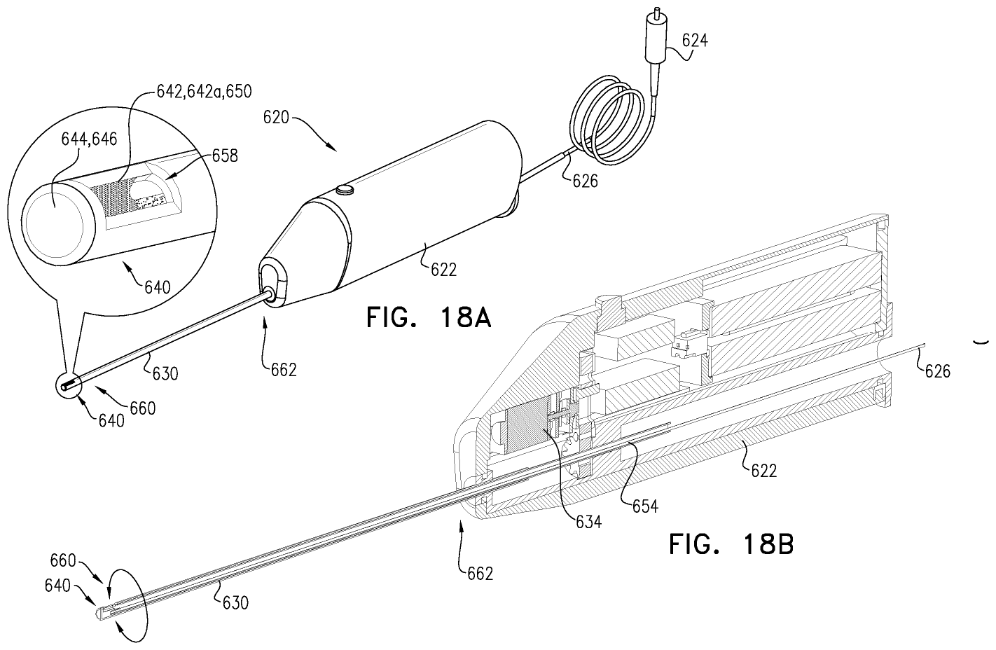

For some applications, and as shown in FIG. 15, motor 534 causes tissue-treatment tip 540, 540′ to move in a reciprocating motion (e.g., a longitudinally reciprocating motion) with respect to the target tissue. For some such applications, motor 534 causes tissue-treatment tip 540, 540′ to longitudinally reciprocate with respect to optical fiber 526 (e.g., to a proximal portion thereof) and/or with respect to shaft 530.

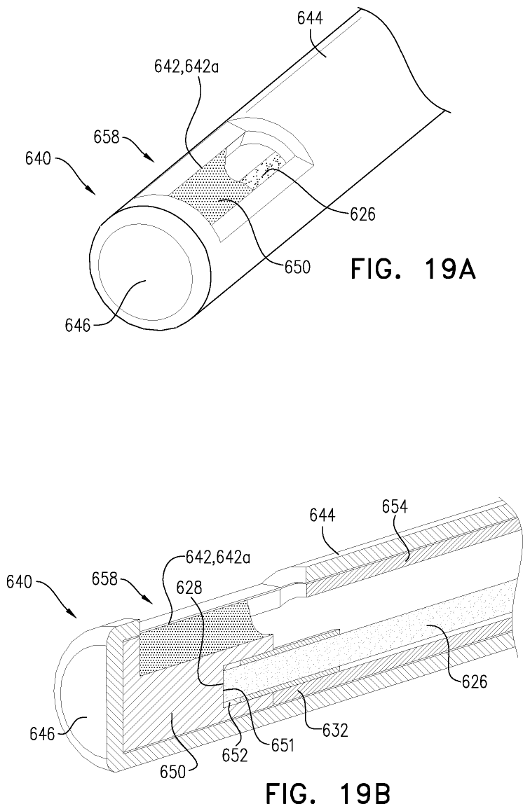

Similarly to as described hereinabove with reference to FIGS. 5A-B, FIGS. 16A-B are side and cross-sectional views of tissue-treatment tip 540 showing positioning of optical fiber 526 in relation to tissue-treatment tip 540 (e.g., to a tissue-contact element 550 thereof) in a manner that facilitates emission of laser energy from the optical fiber (e.g., from distal end 528 thereof) and into the tissue-treatment tip.

For some applications, optical fiber 526 is coupled (e.g., fixedly coupled) to tissue-treatment tip 540. For example, optical fiber 526 may be coupled to tissue-treatment tip 540 according to method 900 described hereinabove. Alternatively or in addition, optical fiber 526 may be coupled to tissue-treatment tip 540 by an epoxy seal that is impermeable to body fluids and saline or other liquids used in a surgical procedure.

For some applications, and as shown, distal end 528 of optical fiber 526 is positioned facing (e.g., near or contacting) a plurality of pores 552 that are etched into a surface of contact element 550. The presence of pores 552 increases a surface area of the portion of the surface of contact element 550 that faces distal end 528 of optical fiber 526. This exemplary embodiment of pores 552 is described hereinabove with reference to FIG. 7A.

It is hypothesized by the inventors that the increased surface area of the portion of the surface of contact element 550 improves efficiency of photothermal conversion, thereby enabling heating the contact element to higher temperatures without increasing the laser energy, while protecting the laser source by limiting backscattering of the laser energy.

For some applications, and as shown, tissue-treatment tip 540 comprises an isolator 532 (e.g., a ceramic isolator) that insulates tissue-contact element 550 from other elements of tissue-treatment device 520, such as tip cannula 554, 554′. In this way, tissue-contact element 550 (e.g., pores 552 thereof) absorbs the laser energy and thermally conducts the absorbed energy toward tissue-treatment tip surface 542, thereby elevating the temperature of the tissue-treatment tip surface. Thus, tissue-treatment tip 540 (i) absorbs laser energy that is emitted from optical fiber 526, and (ii) thermally conducts the absorbed energy to the tissue when tissue-treatment tip surface 542 is placed in contact with the target tissue.

FIGS. 17A-B are side and cross-sectional views of a tissue-treatment tip 540′ that is shaped to define a cavity 552′ having an internal surface 551′. As shown, optical fiber 526′ is positioned to emit laser energy into the cavity (e.g., by the optical fiber being coupled to the cavity). For some applications, and as shown, a portion of internal surface 551′ is a reflective surface 551′a, which reflects the laser energy toward a tissue-treatment tip surface 542′ (e.g., of a tissue-contact element 550′, as shown). At least a portion of internal surface 551′ absorbs the laser energy and thermally conducts the absorbed energy toward tissue-treatment tip surface 542′, thereby elevating the temperature of the tissue-treatment tip surface. In this way, tissue-treatment tip 540′ (i) absorbs laser energy that is emitted from optical fiber 526′, and (ii) thermally conducts the absorbed energy to the tissue when tissue-treatment tip surface 542′ is placed in contact with the target tissue.

Thus, tissue-treatment devices 520, 520′ are used to treat the target tissue by reciprocating motion while tissue-treatment tip surface 542, 542′ is at an elevated temperature due to absorbed thermal energy. For some applications, the use of both absorbed thermal energy and mechanical force reduces an amount of mechanical force that is required for tissue-treatment device 520, 520′ to treat (e.g., to trim a portion of) the tissue. For example, even in the absence of the thermal energy, treatment tips 540, 540′ may trim the tissue to a certain extent if tissue-contact element tissue-contact element 550, 550′ longitudinally reciprocates while tissue-treatment tip surface 542, 542′ contacts the tissue. However, tissue-treatment device 520, 520′ may require a greater mechanical force to trim the tissue without the thermal energy. Thus, the thermal energy facilitates trimming the tissue using a lower mechanical force.

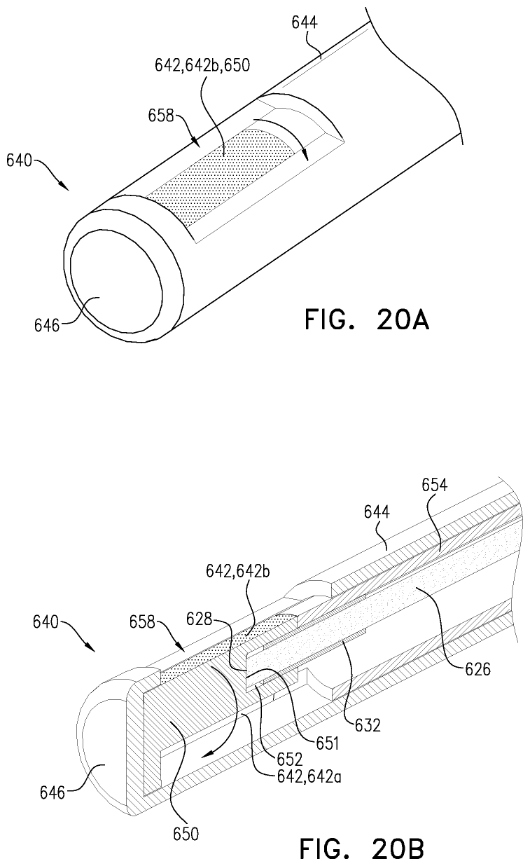

Reference is made to FIGS. 18A-B, 19A-B and 20A-B, which are schematic illustrations showing a tissue-treatment device 620, in accordance with some applications of the invention.

As described hereinbelow, tissue-treatment device 620 comprises a tissue-treatment tip 640 that: (i) conducts heat to the target tissue, and (ii) is operatively coupled to a motor 634 that transfers a mechanical force to the tissue-treatment tip, thereby inducing motion of the tissue-treatment tip. In this way, tissue-treatment device 620 makes use of both thermal energy and mechanical force to treat tissue.

Typically, tissue-treatment device 620 is used with or comprises a laser energy source. FIG. 18A shows tissue-treatment device 620 comprising a laser energy interface 624 that delivers laser energy from the laser energy source, by an optical fiber 626, through an elongate shaft 630 and toward tissue-treatment tip 640 that is disposed at a distal portion 660 of the shaft. Tissue-treatment device 620 is also typically used (e.g., by a surgeon) to treat tissue of a subject, and therefore comprises a handle 622 at a proximal region 662 of shaft 630. Handle 622 is typically used to advance tissue-treatment tip 640 to the target tissue. Some target tissues include tissue of a joint (e.g., a knee, shoulder, hip or spine) such as a bone, a tendon or cartilaginous tissue such as a meniscus. For some applications, a distance from handle 422 to tissue-treatment tip 440, and/or an angular orientation of tissue-treatment tip 640, is fixed with respect to handle 622.

For some applications, and as shown, motor 634 is housed within handle 622. For some such applications, motor 634 is operatively coupled to tissue-treatment tip 640 via a cannula 654 that is disposed within shaft 630. Motor 634 may be operatively coupled to tissue-treatment tip 640 from an alternate location on tissue-treatment device 620 (e.g., the motor may be mounted on shaft 630).

For some applications, and as shown in FIG. 18B, motor 634 causes tissue-treatment tip 640 to move in a reciprocating motion (e.g., a rotationally reciprocating motion) with respect to the target tissue. For some such applications, motor 634 causes tissue-treatment tip 640 to rotationally reciprocate with respect to optical fiber 626 (e.g., to a proximal portion thereof) and/or with respect to shaft 630.

Similarly to as described hereinabove with reference to FIGS. 16A-B, FIGS. 19A-B are side and cross-sectional views of tissue-treatment tip 640 in which a tissue-contact element 650 that has a tissue-treatment tip surface 642 is in a first orientation with respect to a housing 644 (e.g., a window 658 defined thereby) of the tissue-treatment tip. For some applications, and as shown, window 658 is defined in a portion of housing 644 that is proximal of a distal end 646 of the housing.

As shown, tissue-treatment tip surface 642 is shaped to define a blade 642a, which is positioned in FIGS. 19A-B at one side of window 658. In this state, advancing tissue-treatment tip 640 toward the target tissue may result in a portion of the target tissue entering window 658, between blade 642a and housing 644. In this way, longitudinal rotation of tissue-contact element 650 (and therefore, of blade 642a) may result in trimming the portion of the target tissue.

FIGS. 19B and 20B are cross-sectional views of tissue-treatment tip 640 showing positioning of optical fiber 626 in relation to tissue-treatment tip 640 (e.g., to tissue-contact element 650 thereof) in a manner that facilitates emission of laser energy from the optical fiber (e.g., from distal end 628 thereof) and into the tissue-treatment tip. For some applications, and as shown, tissue-treatment tip 640 comprises an isolator 632 (e.g., a ceramic isolator) that insulates tissue-contact element 650 from other elements of tissue-treatment device 620, such as cannula 654.

As shown, distal end 628 of optical fiber 626 faces an internal surface 651 of tissue-contact element 650, such that laser energy emitted from the optical fiber is absorbed by the internal surface. As described hereinabove, the absorbed laser energy undergoes photothermal conversion and is conducted to tissue-treatment tip surface 642.

For some applications, optical fiber 626 is coupled (e.g., fixedly coupled) to tissue-treatment tip 640. For example, optical fiber 626 may be coupled to tissue-treatment tip 640 according to method 900 described hereinabove (e.g., as described hereinabove with reference to FIGS. 7A-D). Alternatively or in addition, optical fiber 626 may be coupled to tissue-treatment tip 640 by an epoxy seal that is impermeable to body fluids and saline or other liquids used in a surgical procedure.

In this way, tissue-contact element 650 absorbs laser energy that is emitted from optical fiber 626, and thermally conducts the absorbed energy toward tissue-treatment tip surface 642 (e.g., toward blade 642a and/or thermal-treatment portion 642b), thereby elevating the temperature of the tissue-treatment tip surface. Thus, tissue-treatment tip 640 thermally conducts the absorbed energy to the tissue when tissue-treatment tip surface 642 is placed in contact with the target tissue.

As described hereinabove, tissue-contact element 650 is operatively coupled to motor 634 (e.g., via cannula 654), such that operating the motor results in rotational reciprocating motion of the tissue-contact element while tissue-treatment tip surface 642 is at an elevated temperature. For some applications, the use of both absorbed thermal energy and mechanical force reduces an amount of mechanical force that is required for tissue-treatment device 620 to treat (e.g., to trim a portion of) the tissue. For example, even in the absence of the thermal energy, treatment tip 640 may trim the tissue to a certain extent if tissue-contact element 650 rotationally reciprocates while tissue-treatment tip surface 642 contacts the tissue. However, tissue-treatment device 620 may require a greater mechanical force to trim the tissue without the thermal energy. Thus, the thermal energy facilitates trimming the tissue using a lower mechanical force.

FIGS. 20A-B are side and cross-sectional views of tissue-treatment tip 640 showing tissue-contact element 650 in a second orientation with respect to window 658 of housing 644, e.g., after rotation of the tissue-contact element. As shown, a thermal-treatment portion 642b of tissue-treatment tip surface 642 is visible through window 658 of housing 644. Similarly to thermal-treatment tip 444 described hereinabove with reference to FIGS. 11A-B, thermal-treatment portion 642b thermally conducts absorbed energy to the target tissue, thereby treating target tissue that the thermal-treatment portion contacts.

Due to the reciprocating movement of tissue-contact element 650, motor 634 may be activated to cause the tissue-contact element to selectively alternate between the first orientation shown in FIGS. 19A-B and the second orientation shown in FIGS. 20A-B, as desired by the operator.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus for use in a surgical procedure, the apparatus comprising:
a tool comprising:
a handle at a proximal region of the tool;
an elongate shaft extending in a distal direction from the handle, the elongate shaft having proximal and distal portions, the distal portion of the shaft being sized and shaped to be inserted into a subject during a surgical procedure;
a tissue-treatment tip disposed at the distal portion of the shaft, the tissue-treatment tip configured to contact tissue of the subject and:
(a) defining a tissue-treatment tip surface that is configured to face the tissue, and
(b) being shaped to define a beam deflector having a deflector surface; and
an optical fiber positioned to emit laser energy into the beam deflector, wherein a first portion of the deflector surface is shaped and positioned so as to reflect the laser energy toward the tissue-treatment tip surface,
wherein a second portion of the deflector surface is configured to absorb the laser energy and thermally conduct the absorbed energy to the tissue-treatment tip surface, and
wherein the tissue-treatment tip surface is configured to thermally conduct the absorbed energy to the tissue by contacting the tissue.

2. The apparatus according to claim 1,
wherein the beam deflector is shaped to define a cavity,
wherein the first portion of the deflector surface is a first portion of an internal surface of the cavity, and
wherein the second portion of the deflector surface is a second portion of the internal surface of the cavity.

3. The apparatus according to claim 1, wherein the first portion of the deflector surface is a reflective coating.

4. The apparatus according to claim 1, wherein the second portion of the deflector surface comprises tungsten.

5. The apparatus according to claim 1, wherein the second portion of the deflector surface comprises molybdenum.

6. The apparatus according to claim 1, wherein the second portion of the deflector surface comprises stainless steel.

7. The apparatus according to claim 1, wherein the second portion of the deflector surface comprises a cobalt-chrome alloy.

8. The apparatus according to claim 1, wherein an angular orientation of the tissue-treatment tip is fixed with respect to an angular orientation of the handle.

9. The apparatus according to claim 8, wherein a distance of the tissue-treatment tip from the handle is fixed.

10. The apparatus according to claim 1, wherein the beam deflector comprises an optical light guide.

11. The apparatus according to claim 10, wherein the optical light guide comprises sapphire or diamond.

12. The apparatus according to claim 10, wherein the optical light guide comprises a material having a melting point that is higher than 1700 degrees Celsius.

13. The apparatus according to claim 1, wherein the optical fiber is a first optical fiber, and the tool further comprises a second optical fiber, each optical fiber being positioned to emit laser energy into a respective region of the beam deflector.

14. The apparatus according to claim 13, wherein the tool is configured such that emitting laser energy into a respective part of the beam deflector causes:
a respective region of the first portion of the deflector surface to reflect the laser energy toward the tissue-treatment tip surface, and
a respective region of the second portion of the deflector surface to absorb the laser energy and thermally conduct the absorbed energy to a respective portion of the tissue-treatment tip surface.

15. The apparatus according to claim 13, wherein the tool further comprises a controller, the controller being configured to separately control:
emission of laser energy from the first optical fiber, and
emission of laser energy from the second optical fiber.

16. The apparatus according to claim 1, wherein the tissue-treatment tip surface comprises a plurality of discrete tissue-contact elements, each of the tissue-contact elements defining a respective tissue-treatment tip surface.

17. The apparatus according to claim 16, wherein the tissue-treatment tip defines a housing that is shaped to define a divider that separates between a first one of the tissue-contact elements and a second one of the tissue-contact elements.

18. The apparatus according to claim 17, wherein the divider comprises an insulating material.
